(19) United States
(12) Patent Application Publication
(10) Pub. No.: US 2014/0238413 A1
(43) Pub. Date: Aug. 28, 2014

(54) NEONATAL THERMAL DEVICE AND RELATED METHOD THEREOF

(71) Applicant: Daniele Ottinger, Charlottesville, VA (US)

(72) Inventor: Daniele Ottinger, Charlottesville, VA (US)

(73) Assignee: UNIVERSITY OF VIRGINIA PATENT FOUNDATION, Charlottesville, VA (US)

(21) Appl. No.: 14/188,217

(22) Filed: Feb. 24, 2014

Related U.S. Application Data
(60) Provisional application No. 61/768,017, filed on Feb. 22, 2013.

(51) Int. Cl. A61B 19/08

(52) U.S. Cl. CPC A61B 19/088; A61B 2019/085
USPC 128/854

(57) ABSTRACT

The neonatal drape device provides protection for a neonatal subject by providing a barrier to the environment that protects the subject from the loss of heat, water and moisture. The neonatal drape device functions as protection primarily by providing a barrier to the environment from the neonatal subject. The neonatal drape device for wrapping a neonatal subject provides the ability of the neonatal drape device to be repeatably self-adhesive and repeatably wrap around medical devices, tools, instruments, implements, parts of anatomy, or any other object protruding from the inside of the neonatal drape device to the outside of the neonatal drape device. The pliable nature of the material allows it to conform to the protruding object or a portion of anatomy, providing protection to the neonatal subject while also allowing repeatable access to the neonatal subject by the protruding object, or the uncovering of select portions of anatomy.

### CROSS-REFERENCE TO RELATED APPLICATIONS

The present application claims benefit of priority under 35 U.S.C. §119(e) from U.S. Provisional Application Ser. No. 61/768,017, filed Feb. 22, 2013, entitled “Neonatal Thermoregulation Drape and Related Method;” the disclosure of which is hereby incorporated by reference herein in its entirety.

### TECHNICAL FIELD

The present invention relates generally to the field of neonatal drape devices and the related method of use and manufacture. More specifically, the invention is in the subfield of thermally regulating the temperature of the neonatal subjects.

### BACKGROUND

Neonatal skin care, for example, is of particular importance when caring for infants in the Neonatal Intensive Care Unit (NICU). Infants born at 30 weeks gestation or less have a higher risk for developing hypothermia in the delivery room and in the NICU just prior to being placed in a heated and humidified incubator. The extremely premature infant also has an increased risk for developing cold stress due to insufficient subcutaneous fat, decreased brown fat storage, thin immature skin and large body surface to mass ratio. Hypothermia increases the risk for morbidity and mortality in these preterm infants, where moderate to severe hypothermia (32.0 to 35.9° C.) has been associated with Intraventricular Hemorrhage (IVH) and death. Hypothermia can also lead to respiratory distress, hypoxia, hypoglycemia, metabolic acidosis, renal failure, coagulopathy and Necrotizing Enterocolitis (NEC).

These premature babies are also persistently in an extended posture, which predisposes them to increased Trans-Epidermal Water Loss (TEWL) and heat loss. At birth, infants are placed on a warming mattress that is placed under a radiant heater. Radiant warmers are a good source for providing heat to these infants, but they have also been linked to an increase in insensible water loss due to evaporation and convective heat loss from the ambient air moving over their bodies.

### Overview

After delivery, the neonatal subject is exposed to a sudden change of temperature from the interior of the mother, at approximately normal body temperature, to a delivery room that is typically 25 degrees Fahrenheit cooler. The neonatal subject, being born prematurely, is not yet able to regulate its own body temperature, and must be kept warm until it can be placed into an incubator. It is also necessary to keep the neonatal subject moist, and it is generally preferred that the neonatal subject should remain in a coating of amniotic fluid, if possible. However, the transfer to an incubator may not be immediate, and the neonatal subject must be protected to prevent loss of heat and moisture.

As the neonatal subject is very vulnerable at this stage of life, an aspect of an embodiment of the present invention device provides for protecting the subject that not only prevents loss of heat and moisture, but also allows for access to the neonatal subject for examinations or medical procedures. It should be appreciated that an aspect of an embodiment of the present invention device may be used in conjunction with thermal radiant heaters (e.g., a radiant warmer or the like), so it is preferable that the device prevents heat loss through convection and conduction, but still allows thermal radiation to pass through and warm the neonatal subject. It is preferable that an embodiment of the present invention device allow for visual inspections of the neonatal subject, to observe the color and condition of the subject, and to visually confirm that the subject is breathing. It is preferable that an aspect of an embodiment of the present invention device allow for auscultation without removal of the device from the neonatal subject. Finally, it is preferable that an aspect of the present invention device be sufficiently gentle on the subject that it will not harm their skin, which may be very fragile and, in some cases, consist of only a single layer of cells.

An aspect of an embodiment of the present invention neonatal drape device relates to the care of premature babies immediately after birth. The device provides a barrier to the outside world for the neonatal subject, preventing the majority of conduction and convection of heat away from the subject into the air. In particular, the neonatal drape device will prevent drafts from cooling the baby. The device may also help seal the neonatal subject in amniotic fluid, preventing moisture loss and appreciable drying of the subject. However, the device will allow for the use of thermal radiation heaters that are routinely used to keep the subject warm. It should also be appreciated that the neonatal drape device is sufficiently thin to allow auscultation there through, and that an aspect of an embodiment of the device is a material that is transparent or translucent to allow for visual inspection of color, condition, and breathing function.

An aspect of an embodiment of the present invention neonatal drape device is configured in such a way, through the use of separation lines, to allow a user to open selected spans of the device to create an access passage to the neonatal subject in the region of choice. Common areas of access may include, but are not limited to, the umbilicus and limbs of the neonatal subject. The neonatal drape device is configured so that the user may initially wrap the neonatal subject and then, after wrapping, selectively tear separation lines to gain access to the desired portion of the subject.

It should be noted that an aspect of an embodiment of the present invention is comprised of a material that is repeatably self adhesive due to the characteristics of the material itself. The device is devoid of mechanical fasteners or adhesives that would pose potential dangers to the delicate skin of a neonatal subject. Also, because the material is repeatably self adhesive, it will adhere to itself allowing for effective wrapping of the neonatal subject without significant gaps in the wrapping. This provides effective protection to the neonatal subject, and facilitates ease of use by the user. It should be appreciated that the neonatal drape device does not significantly adhere to the skin of the neonatal subject.

An aspect of an embodiment of the present invention neonatal drape device provides further benefits in the care of a neonatal subject because, due to the repeatable self adhesive nature of the material and its ability to conform to a variety of geometries, it may be re-wrapped by the user to close off the access passage. The self adhesion will allow the span that has been opened to re-adhere to other portions of the device and provide adequate protection to the newborn. Furthermore, the ability of the neonatal drape device to conform to a number of geometries allows the span to close off exposure of the subject to the air, while still allowing for a medical device, tool, instrument, or implement to protrude from the interior of the wrap to the exterior. This is useful if the neonatal subject requires, for example, intravenous injections or for blood samples to be taken. It should be appreciated that this ability to conform to a number of shapes and geometries also allows the user to selectively expose a portion of anatomy by allowing it to protrude from the wrap, as the case may be for a limb or the umbilicus.

An aspect of an embodiment of the present invention provides, but not limited thereto, a neonatal drape device for protecting a neonatal subject. The device may comprise: a sheet configured to receive the neonatal subject in a supine position having at least one side edge and a plurality of separation lines; the separation lines extending inwardly from the at least one side edge of the sheet; the sheet being sufficiently pliable so as to wrap and conform to the neonatal subject; the sheet comprised of a material that is repeatably self adhesive as wrapped around the neonatal subject, wherein the repeatable self adhesion is due to characteristics of the material; the separation lines configured to define a span between adjacent the separation lines or between the separation line and the at least one side edge to allow user to selectively separate the span from an adjacent span in its wrapped configuration and form an access passage to the neonatal subject; and the span configured to repeatably self adhere to the sheet in its wrapped configuration to allow the user to selectively close the access passage.

An aspect of an embodiment of the present invention provides, but not limited thereto, a neonatal drape device for protecting a neonatal subject. The device may comprise: a sheet configured to receive the neonatal subject in a supine position having at least one side edge and a plurality of separation lines; the separation lines extending inwardly from the at least one side edge of the sheet from a first portion and a second portion of the sheet, wherein the first and second portions are generally opposed; the sheet being sufficiently pliable so as to wrap and conform to the neonatal subject; the sheet comprised of a material that is repeatably self adhesive as wrapped around the neonatal subject, wherein the repeatable self adhesion is due to characteristics of the material; wherein for each of the first portion of the sheet and the second portion of the sheet, the separation lines configured to define a span between adjacent separation lines or between the separation line and the at least one side edge to allow user to selectively separate the span from an adjacent span in its wrapped configuration and form an access passage to the neonatal subject; and wherein for each of the first portion of the sheet and the second portion of the sheet, the span configured to repeatably self adhere to the sheet in its wrapped configuration to allow the user to selectively close the access passage.

An aspect of an embodiment of the present invention provides, but not limited thereto, a method of protecting a neonatal subject. The method may comprise: providing a sheet to receive the neonatal subject, the sheet having at least one side edge and a plurality of separation lines; placing the neonatal subject in a supine position on the sheet; wrapping the neonatal subject with the sheet so as to conform the sheet to the neonatal subject and adhering portions of the sheet to other portions of the sheet; tearing along the separation lines, after the neonatal subject has been wrapped, releasing a span between the separation lines allowing access to the neonatal subject; and re-applying the span thereby closing the access and re-adhering the span to the sheet.

The neonatal drape device provides protection for a neonatal subject by providing a barrier to the environment that protects the subject from the loss of heat, water and moisture. The neonatal drape device functions as protection primarily by providing a barrier to the environment from the neonatal subject. The neonatal drape device for wrapping a neonatal subject provides the ability of the neonatal drape device to be repeatably self-adhesive and repeatably wrap around medical devices, tools, instruments, implements, parts of anatomy, or any other object protruding from the inside of the neonatal drape device to the outside of the neonatal drape device. The pliable nature of the material allows it to conform to the protruding object or a portion of anatomy, providing protection to the neonatal subject while also allowing repeatable access to the neonatal subject by the protruding object, or the uncovering of select portions of anatomy.

These and other objects, along with advantages and features of various aspects of embodiments of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 provides a schematic illustration (plan view) of an aspect of embodiment of the neonatal drape device.

FIG. 2A provides a schematic illustration (plan view) of an aspect of embodiment of the neonatal drape device.

FIG. 2B provides a side schematic illustration of an aspect of an embodiment of the neonatal drape device, as shown in FIG. 2A for example.

FIG. 2C provides a side schematic illustration of an aspect of an embodiment of the neonatal drape device, as shown in FIG. 2B for example, with the device being shown in a wrapped configuration.

FIG. 2D provides a side schematic illustration of an aspect of embodiment of the neonatal drape device, as shown in FIG. 2C for example, after the device has been in its wrapped configuration; whereby the device is now illustrated after the user (not shown) selectively separates one or more of the spans from its adjacent spans and the other respective portions of the sheet.

FIG. 2E provides a side schematic illustration of an aspect of embodiment of the neonatal drape device, as shown in FIG. 2D for example, after the part of the device had been in an open configuration (partially unwrapped); whereby the device is now illustrated after the user (not shown) selectively closes spans (returns or reapplies) of the sheet to re-adhere the spans to the wrapped sheet 14 in the wrapped configuration.

FIG. 3 provides a schematic illustration (perspective view) of an aspect of an embodiment of the present invention drape device whereby a neonatal subject is laying in supine position on the device.

FIG. 4 provides a schematic illustration (perspective view) of an aspect of an embodiment of the present invention drape device as shown in FIG. 3, whereby the neonatal subject is lying in supine position and has been wrapped consistent with the teachings of various embodiments of the present invention disclosed herein.

FIG. 5A provides a schematic illustration (perspective overhead view) of an aspect of an embodiment of the neonatal drape device.

FIG. 5B provides a schematic illustration (perspective overhead view) the neonatal drape device as shown in FIG. 5B with the addition that the left portion of the device comprising of associated spans has been wrapped over the neonatal subject.

FIG. 5C provides a schematic illustration (perspective overhead view) the neonatal drape device as shown in FIG. 5B with the addition that the right portion of the device comprising of spans has been wrapped over the already wrapped left portion so as to completely wrap the neonatal subject.

FIG. 5D provides a schematic illustration (perspective overhead view) of the neonatal drape device as shown in FIG. 5C with the addition that the user (not shown) has selectively separated one of the spans away from its adjacent spans and the right portion of the sheet while the device is in its wrapped configuration; and the user (not shown) has selectively separated one of the other spans away from its adjacent spans and the neonatal subject and left portion of the sheet while the device is in its wrapped configuration.

FIG. 6 provides a schematic illustration (perspective overhead view) of an aspect of an embodiment of the neonatal drape device as shown in FIG. 5A, except for the variation that one or more or all of the separation lines may traverse completely across the device.

FIG. 7 provides a schematic illustration (perspective overhead view) of an aspect of an embodiment of the neonatal drape device as shown in FIG. 5A, except for the variation that one or more or all of the spans may be configured so as to have a tabbed shape along the edge of the device.

FIG. 8A provides a schematic illustration of an aspect of an embodiment of the neonatal drape device with the objective of showing a side view of a portion of the edge of the sheet.

FIG. 8B provides a schematic illustration of an aspect of an embodiment of the neonatal drape device of the embodiment shown in FIG. 8A, except for the variation of the spans having a generally curved profile rather than generally flat.

FIG. 8C provides a schematic illustration of an aspect of an embodiment of the neonatal drape device of the embodiment shown in FIG. 8A, except for the variation of the spans having a generally horseshoe or mushroom profile rather than generally flat.

FIG. 8D provides a schematic illustration of an aspect of an embodiment of the neonatal drape device of the embodiment shown in FIG. 8A, except for the variation of the spans having a greater width and overlapping each other.

### DETAILED DESCRIPTION OF EXEMPLARY EMBODIMENTS OF THE PRESENT INVENTION

It should be appreciated that an aspect of an embodiment of the neonatal drape device for wrapping a neonatal subject is the ability of the neonatal drape device to wrap around medical devices, tools, instruments, implements, parts of anatomy, or any other object protruding from the inside of the neonatal drape device to the outside of the neonatal drape device. The pliable nature of the material allows it to conform to the protruding object or a portion of anatomy, providing protection to the neonatal subject while also allowing access to the neonatal subject by the protruding object, or the uncovering of select portions of anatomy. For example, a user may initially wrap the neonatal subject in the neonatal drape device. After the initial wrap, the user may release a span by tearing along separation lines to create an access passage. The user then may insert a medical device, tool, instrument, implement, or his or her hands through the access passage. Alternatively, a portion of the anatomy of the neonatal subject may be extended so as to protrude through the access passage. The user may then reapply the separated span in such a way as to conform to the contours of the neonatal subject (e.g., patient) and also conform to the contours of the protruding medical device, tool, instrument, implement, or exposed portion of anatomy. The neonatal drape device thus allows the user to maintain coverage of the neonatal subject while also maintaining the required access.

An aspect of an embodiment of an embodiment of the neonatal drape device provides protection for a neonatal subject by providing a barrier to the environment that protects the subject from the loss of heat, water and moisture. The neonatal drape device functions as protection primarily by providing a barrier to the environment from the neonatal subject. As a barrier, the neonatal drape device significantly limits the amount of air that may come into contact with the neonatal subject, and it prevents drafts from contacting the neonatal patient. Because of this, evaporation of fluids from the neonatal patient's skin is significantly reduced, as is heat loss through conduction and convection with the surrounding air. This is very important as the neonatal patient has recently been introduced into an environment significantly colder than the environment inside the mother.

Another advantage of an aspect of embodiment of the present neonatal drape device is that, while reducing heat loss from convection and conduction, it does not significantly block thermal radiation. While thermal radiation plays only a small role in the amount of heat lost by a neonatal subject, radiant heat sources are used to keep the subject warm after birth and before the introduction to an incubating unit. It is possible to use the neonatal drape device in conjunction with a radiant heat source to keep the neonatal patient warm. Furthermore, the neonatal drape device may be sufficiently unobtrusive to thermal radiation, wherein sensors used in conjunction with radiant heat sources and the neonatal drape device remain functional and prevent overheating of the neonatal subject. These sensors (e.g., temperature probe or the like) are typically placed on the abdomen of the neonatal subject in order to detect the amount of radiant heat that the subject is exposed to. Traditional blankets will block these sensors, rendering them non-functional.

Yet another advantage of an aspect of an embodiment of the present neonatal drape device is that, particularly when configured to be transparent, the neonatal drape device allows for examinations of the neonatal subject while still wrapped in the neonatal drape device. In an aspect of an embodiment of the neonatal drape device, the device is composed of a material that is sufficiently thin to allow the user to perform an examination of the neonatal subject, including the ability to perform auscultation of the neonatal subject. Furthermore, in an embodiment of the neonatal drape device, when it is composed of a material that is sufficiently transparent or translucent, the user may visually examine the neonatal subject to see condition, color, or breathing function.

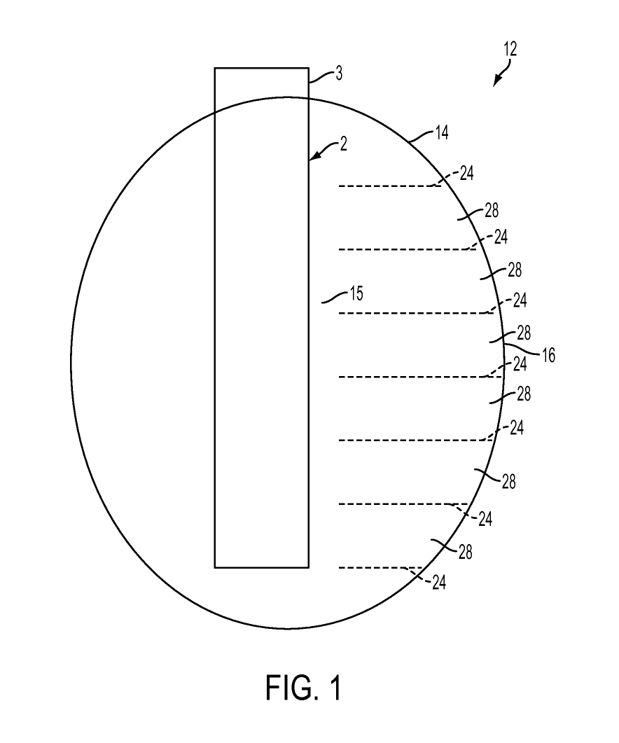

FIG. 1 provides a schematic illustration (plan view) of an aspect of embodiment of the neonatal drape device 12 for protecting a neonatal subject 2. The device 12 may comprise a sheet 14 configured to receive the neonatal subject 2 in a supine position and having at least one side edge 16 along with a plurality of separation lines 24. The neonatal subject 2 may be initially received on the generally central region 15 of the sheet 14; and whereby the head 3 of the subject 2 is schematically illustrated located generally beyond the edge 16 of the sheet 14. The separation lines 24 may extend inwardly from the side edge 16 of sheet 14. The sheet 14 may be sufficiently pliable so as to wrap and conform to the neonatal subject 2. The sheet 14 may be comprised of a material that is repeatably self adhesive as wrapped around the neonatal subject 2, wherein said repeatably self adhesion is due to characteristics of the material itself. The separation lines may be configured to define a span 28 between various adjacent separation lines 24 to a allow user (not shown) to selectively separate one or more of the spans 28 from an opposing portion of the sheet 14 while the device is in its wrapped configuration and thereby form an access passage 32 or passages 32 (not shown) to the neonatal subject 2 (while in the wrapped configuration). The spans 28 are configured to repeatably self adhere to the sheet in its wrapped configuration to selectively allow the user (not shown) to close the access passage(s) 32 (not shown) after it has been opened.

Still referring to FIG. 1, the material of sheet 14 may be a variety of materials including, but not limited thereto, any combination of one or more of the following: polymer, polyethylene, polyurethane, vinyl, Poly vinyl chloride (PVC), rubber, or vinyl or the like. The material of sheet 14 may be a variety of materials including, but not limited thereto, any combination of one or more of the following: breathable material, permeable material, impermeable material, micro-porous material, or nano-porous material. It should be appreciated that the material may be selected to be any material that performs the function of self-adhesion (as well as other attributes of an aspect of an embodiment as disclosed herein).

It should be appreciated that the material may be selected to be any material or thickness that provides for the function of self-adhesion, pliability, visual inspection, thermal protection, and/or ability to perform auscultation (as well as other attributes of an aspect of an embodiment as disclosed herein).

Still referring to FIG. 1, the material of the sheet 14 may be transparent. The sheet 14 may be thin enough to allow auscultation there through.

Still referring to FIG. 1, the separation lines are configured to remain un-separated during the initial wrapping of the neonatal subject 2 by the user (not shown) and to separate upon the user selectively separating one or more of the spans from one another while the sheet 14 is in its wrapped configuration.

Still refereeing to FIG. 1, the separation lines 24 may comprises at least one of the following so as to allow for separation along defined lines: perforations, scores, thin portions, or weak portions or the like.

Still referring to FIG. 1, the device 12 may further comprise an appendage (not shown). The appendage (not shown) may serve as a hood for head 3 of the neonatal subject 2. Similarly, the appendage or other appendages (not shown) may be provided to accommodate legs, arms, hands, and/or feet or the like.

Still referring to FIG. 1, the device 12 may be generally circular as shown with one edge; or may be elliptically shaped. Alternatively, it may have two or more edges or may be any polygonal shape, such as a rectangle, square, triangle, hexagon, pentagon, octagon, etc. The sides may be uniform or vary (or some combination).

It should be appreciated that the various embodiments of device disclosed herein may be sterilized if desired, required or needed, which could be critical under certain circumstances and instances.

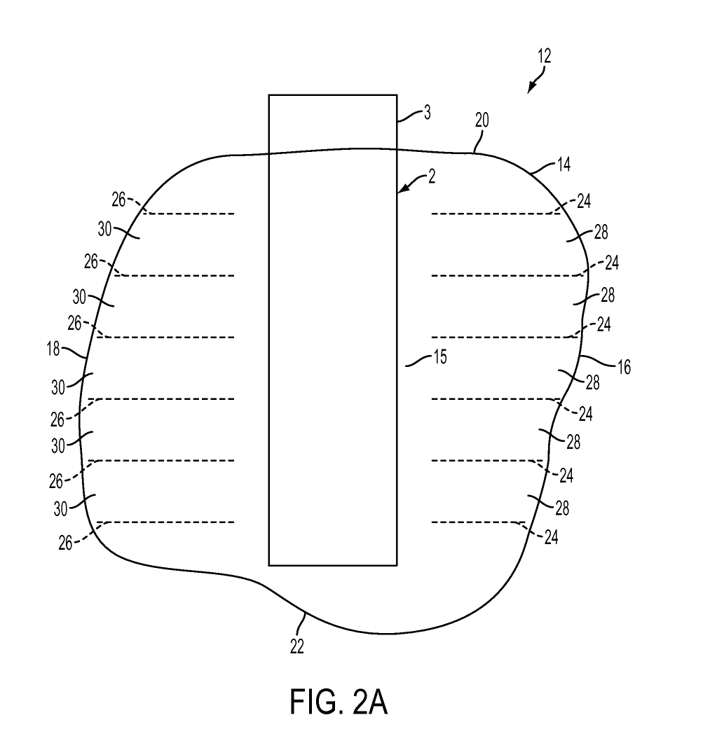

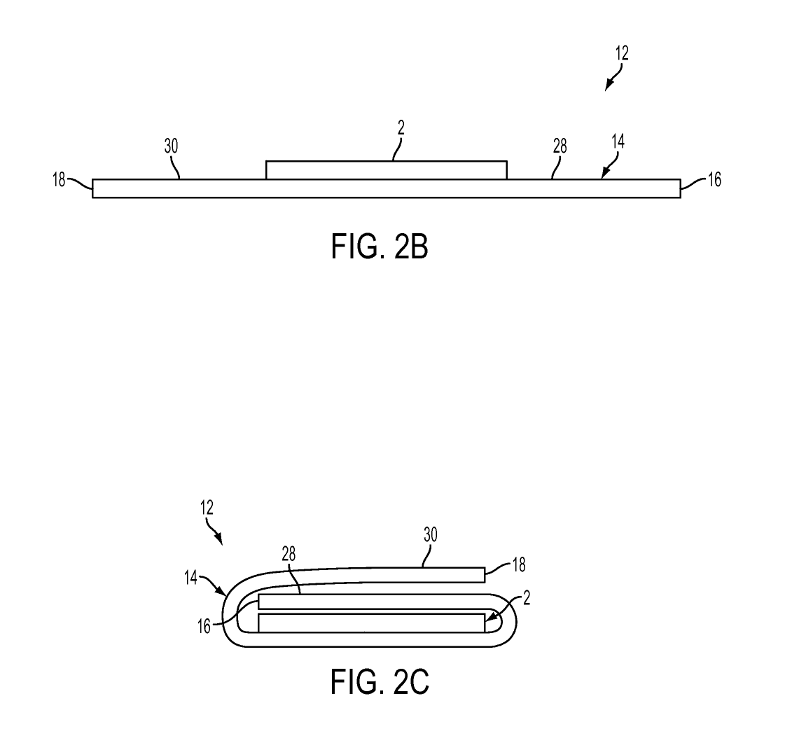

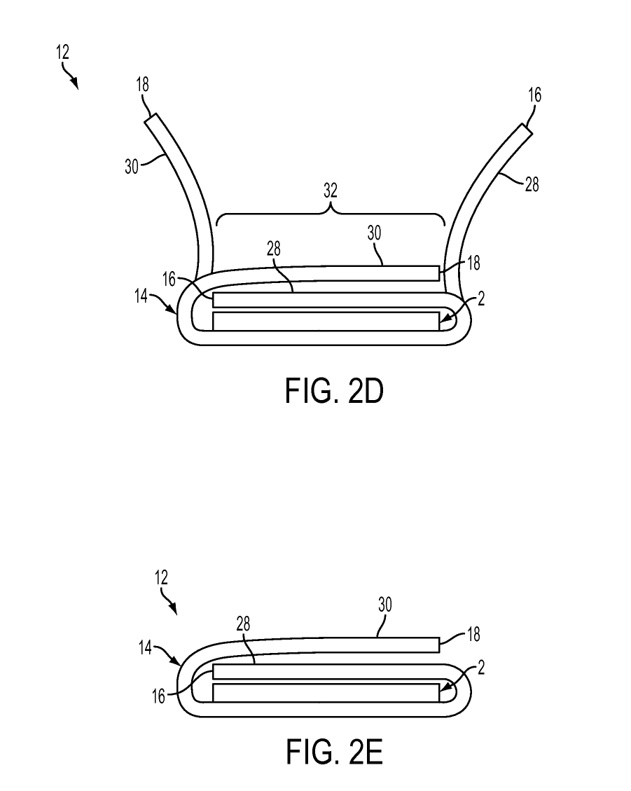

FIG. 2A provides a schematic illustration (plan view) of an aspect of an embodiment of the neonatal drape device 12 for protecting a neonatal subject 2. The device 12 may comprise a sheet 14 configured to receive the neonatal subject 2 in a supine position and having four edges 16 (left), 18 (right), 20 (upper), 22 (lower) along with a plurality of separation lines 24 (left side), 26 (right side). The neonatal subject 2 may be initially received on the generally central region 15 of the sheet 14; and whereby the head 3 of the subject 2 is schematically illustrated located generally beyond the edge 20 of the sheet 14. The separation lines 24, 26 may extend inwardly from the side edges 16, 18 of sheet 14. The sheet 14 may be sufficiently pliable so as to wrap and conform to the neonatal subject 2. The sheet 14 may be comprised of a material that is repeatably self adhesive as wrapped around the neonatal subject 2, wherein said repeatable self adhesion is due to characteristics of said material itself. The separation lines may be configured to define a span 28 (left), 30 (right) between various adjacent separation lines 24, 26 to a allow user (not shown) to selectively separate one or more of the spans 28, 30 from an opposing portion of the sheet 14 while the device is in its wrapped configuration and thereby form an access passage 32 or passages 32 (not shown) to the neonatal subject 2 (while in the wrapped configuration). The spans 28, 30 are configured to repeatably self adhere to other spans of the opposing portions of the sheet in its wrapped configuration to selectively allow the user (not shown) to close the access passage(s) 32 (not shown) after it has been opened.

FIG. 2B provides a side schematic illustration of an aspect of an embodiment of the neonatal drape device 12, as shown in FIG. 2A for example, for protecting a neonatal subject 2. The device 12 may comprise a sheet 14 configured to receive the neonatal subject 2 in a supine position and having two side edges 16, 18 with respective spans 28, 30.

FIG. 2C provides a side schematic illustration of an aspect of an embodiment of the neonatal drape device 12, as shown in FIG. 2B, for example, with the sheet 14 being shown in a wrapped configuration whereby the portions of the sheet 14 having spans referenced as 28 are wrapped in contact with the neonatal subject 2 and the portions of the sheet 14 having spans referenced as 30 are wrapped upon the portion of the sheet having spans referenced as 28. It should be appreciated the order of the wrapping of the respective portions may be reversed.

FIG. 2D provides a side schematic illustration of an aspect of embodiment of the neonatal drape device 12, as shown in FIG. 2C, for example, after the sheet 14 has been in its wrapped configuration; whereby the sheet is now illustrated (FIG. 2D) after the user (not shown) selectively separates one or more of the spans 28, 30 from the other respective spans or portions of the sheet 14, thereby creating an access passage 32 or passages 32 to the neonatal subject 2. For instance, the portions of the sheet 14 having spans referenced as 30 are un-wrapped away from (or substantially removed from or directed away from) the portion of the sheet having spans referenced as 28 and adjacent spans; and the portions of the sheet 14 having spans referenced as 28 are un-wrapped from being in contact with the neonatal subject 2. As shown in the drawing, a set of spans 28, 30 closest to the view point is in the wrapped configuration; and a set of spans 28, 30 beyond these closed spans 28, 30 (relative to the view point) is in the unwrapped (open) configuration allowing for the access as desired or required. The access passage 32 allows access by the user (not shown) to any existing medical device, tool, instrument, implement, or exposed portion of anatomy; as well as providing access clearance to insert or attach any medical device, instrument, tool, instrument, or implement in communication with the neonatal subject, all while the device remains in the wrapped configuration.

FIG. 2E provides a side schematic illustration of an aspect of embodiment of the neonatal drape device 12, as shown in FIG. 2D, for example, after the spans 28, 30 had been in an open configuration (partially unwrapped portion); whereby the sheet is now illustrated after the user (not shown) selectively closes spans 28, 30 (returns or reapplies) of the sheet 14 to re-adhere the spans to the wrapped sheet 14 in the wrapped configuration to protect the neonatal subject 2. As disclosed herein, the sheet and spans are pliable and flexible so as to be re-applied as desired or required to accommodate any particular anatomy features or objects (e.g., to accommodate protrusions, curvatures, structures, etc.). It should be appreciated that the device re-adheres to itself due to the characteristics of the material itself. As such, it does not require tape, structures or adhesives to re-seal or re-wrap the device.

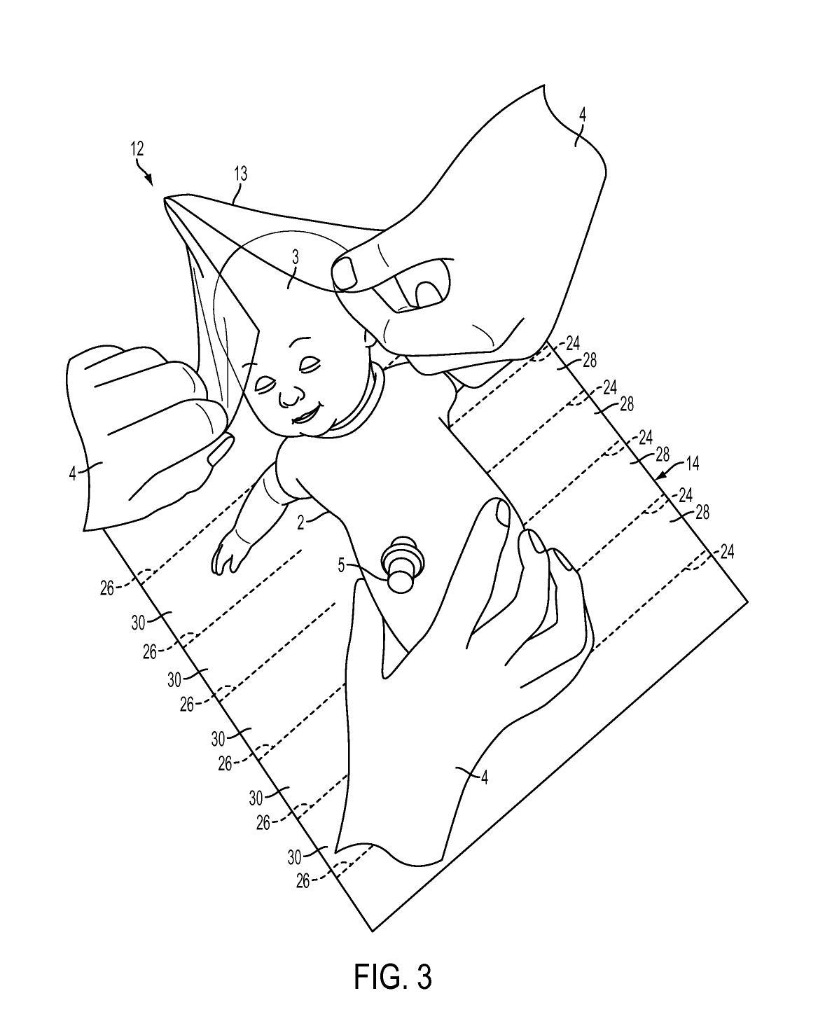

FIG. 3 provides a schematic illustration (perspective view) of an aspect of an embodiment of the present invention drape device 12 whereby a neonatal subject 2 is laying in supine position on the sheet 14. Hands of users 4, such as physicians, nurses, clinicians, or care providers are shown. Also shown is a hood 13 of the device 12 being place over the head 3 of the subject 2. Finally, an umbilical cord stump 5 is visible. As disclosed herein, the sheet 14 is comprised of a material that is repeatably self adhesive that will eventually be wrapped around the neonatal subject 2, wherein said repeatable self adhesion is due to characteristics of the material itself. The sheet 14 having separation lines 24, 26 configured to define respective spans 28 on the left portion of the sheet 14 and spans 30 on the right portion of the sheet 14 between the various adjacent separation lines 24, 26.

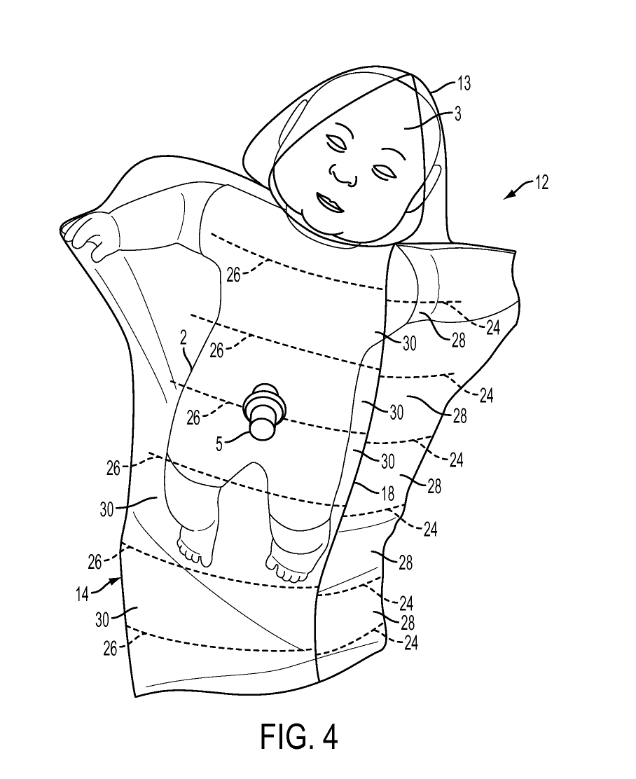

FIG. 4 provides a schematic illustration (perspective view) of an aspect of an embodiment of the present invention drape device 12 as shown in FIG. 3, whereby the neonatal subject 2 is laying in supine position and has been wrapped consistent with the teachings of various embodiments of the present invention disclosed herein. For instance, the spans 28, 30 having separation lines 24, 26 are configured to repeatably self adhere to other spans of the opposing portions of the sheet 14 to provide its wrapped configuration. In particular, as generally illustrated, the portions of the sheet 14 comprising of spans referenced as 30, having a side edge referenced as 18, are adhered on top of the portions of the sheet 14 comprising spans referenced as 28. As shown, a majority of the area that includes the spans referenced as 28 are underneath the spans referenced as 30 (it should be appreciated that the order of top and bottom could be reversed). Also shown is the umbilical cord stump 5 that protrudes from the device after an access passage had been to created (to provide access and clearance) consistent with the teachings of the present invention disclosed herein.

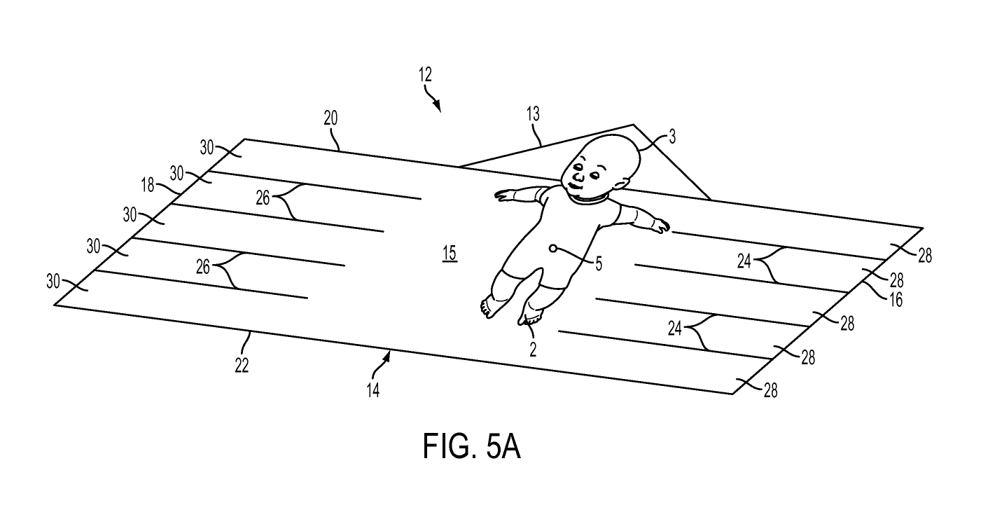

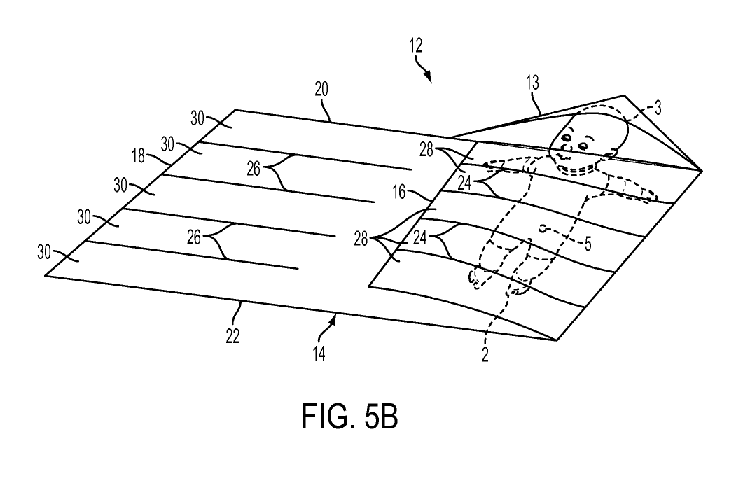

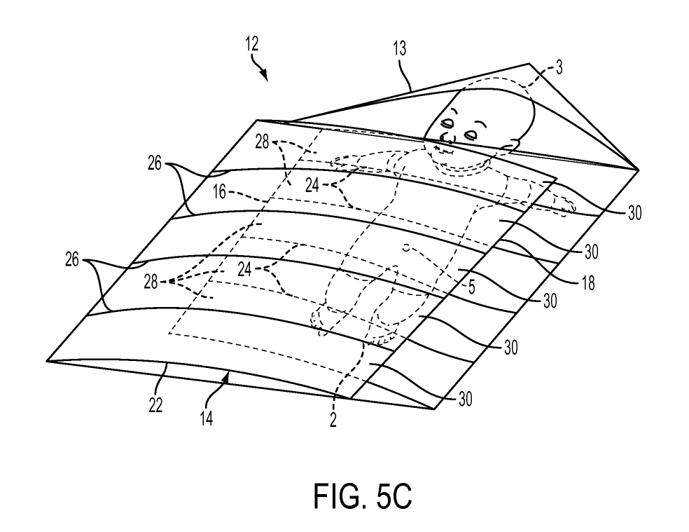

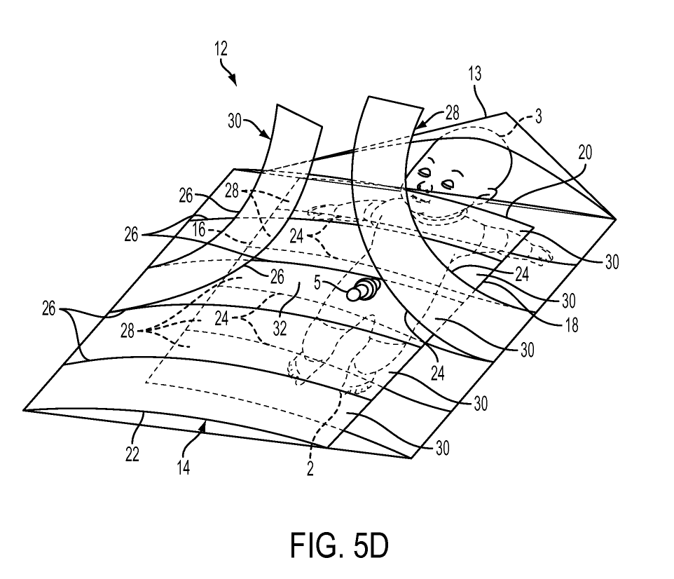

FIG. 5A provides a schematic illustration (perspective overhead view) of an aspect of an embodiment of the neonatal drape device 12 for protecting a neonatal subject 2. The device 12 may comprise a sheet 14 configured to receive the neonatal subject 2 in a supine position and having four edges 16 (left), 18 (right), 20 (upper), 22 (lower) along with a plurality of separation lines 24 (left side), 26 (right side). As shown in the current illustration, the sheet 14 has rectangular shape. The neonatal subject 2 may be initially received on the generally central region 15 of the sheet 14; and whereby the head 3 of the subject 2 is schematically illustrated located generally beyond the edge 20 of the sheet 14. As shown in the current illustration, the head 3 is set upon the outside of the hood 13. The separation lines 24, 26 may extend inwardly from the side edges 16, 18 of sheet 14. The sheet 14 may be sufficiently pliable so as to wrap and conform to the neonatal subject 2. The sheet 14 may be comprised of a material that is repeatably self adhesive as wrapped around the neonatal subject 2, wherein said repeatable self adhesion is due to characteristics of said material itself. The separation lines may be configured to define a span 28, 30 between various adjacent separation lines 24, 26. Near the top or bottom edges, for example, a span could be defined between the top edge and the adjacent separation line (rather than between two separation lines).

FIG. 5B provides a schematic illustration (perspective overhead view) the neonatal drape device 12 as shown in FIG. 5B with the addition that the left portion of the sheet 14 comprising of associated spans 28 has been wrapped over the neonatal subject 2. As shown in the current illustration, the head 3 is set inside of the hood 13. It should be appreciated that the shape of the hood may be any chosen configuration so to be able to cover the head as desired or required. The hood may be cup shaped to accommodate the head or may be single layer to be folded or wrapped over the head.

FIG. 5C provides a schematic illustration (perspective overhead view) the neonatal drape device 12 as shown in FIG. 5B with the addition that the right portion of the sheet 14 comprising of spans 30 has been wrapped over the already wrapped left portion (comprising of spans 28) so as to completely wrap the neonatal subject 2.

FIG. 5D provides a schematic illustration (perspective overhead view) of the neonatal drape device 12 as shown in FIG. 5C with the addition that the user (not shown) has selectively separated one of the spans 30 away from the right portion of the sheet 14 and its respective adjacent spans while the device is in its wrapped configuration. Moreover, the user (not shown) has selectively separated one of the spans 28 away from its respective adjacent spans and the neonatal subject 2 and the left portion of the sheet 14 while the device (e.g., sheet 14) is in its wrapped configuration; and thereby forming an access passage 32 or passages (i.e., clearance) to the neonatal subject 2 while the balance of the sheet 14 continues to remain in the wrapped configuration. As illustrated, the umbilical cord 5 and other surrounding areas/regions of the neonatal subject 2 are exposed. Depending on which spans and how many are lifted or moved will determine how much and what location of the neonatal subject 2 will be exposed for access, etc. Another factor of area of exposure is dependent on the area of the spans, which may vary as desired or required. The access passage 32 allows access by the user (not shown) to any existing medical device, tool, instrument, implement, or exposed portion of anatomy; as well as providing access clearance to insert or attach any medical device, instrument, tool, instrument, or implement in communication with the neonatal subject. It should be appreciated that various types of medical devices, tools, instruments, or implements may be in communication with the umbilical cord 5, as well as any other anatomy or area of the neonatal subject 2 as desired or required. The spans 28, 30 are configured to repeatably self adhere to the sheet 14 in its wrapped configuration to selectively allow the user (not shown) to close the access passage(s) 32 (not shown) after it has been opened. The opening, closing and self re-adhering may be repeated numerous times as desired, required, and/or needed. Accordingly, the sheet 14 may return to the wrapped configuration as shown in FIG. 5C, with the exception, for example, that the umbilical cord 5 may protrude beyond the sheet 14 as the spans 28, 30 are re-wrapped flexibly and conveniently around the umbilical cord 5. It should be appreciated that any one or more (as well as all) of the spans 28, 30 may be selectively separated and re-wrapped as desired, required, or needed.

It should be appreciated that the device re-adheres to itself due to the characteristics of the material itself. As such, it does not require tape, structures or adhesives to re-seal or re-wrap the device.

It should be appreciated, an area of exposure or access may be dependent upon how much of the separation line is separated from adjacent spans. For instance, the entire separation line may be separated or only a portion of the separation line may be separated. Moreover, the separation line may be partially separated (so as to create a slit or the like) at its interior region or at commencing at the edge of the device, as desired or required.

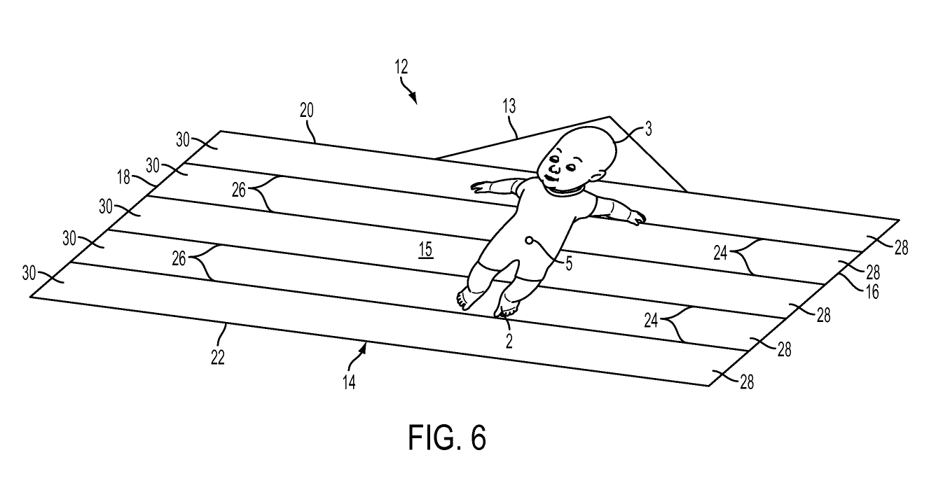

FIG. 6 provides a schematic illustration (perspective overhead view) of an aspect of an embodiment of the neonatal drape device 12 as shown in FIG. 5A, except for the variation that one or more or all of the separation lines 24, 26 may traverse completely across the sheet 14.

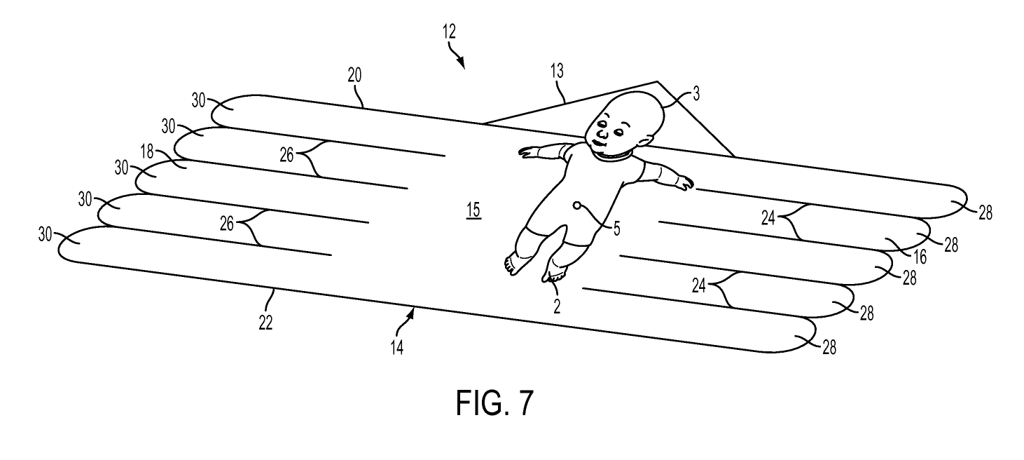

FIG. 7 provides a schematic illustration (perspective overhead view) of an aspect of an embodiment of the neonatal drape device 12 as shown in FIG. 5A, except for the variation that one or more or all of the spans 28, 30 may be configured so as to have a tabbed shape along the edge of the sheet 14. The tabs may include any variety of shapes including curved, rectangular, or triangular, etc. This may help the user to hold, grip or retain a span, for instance.

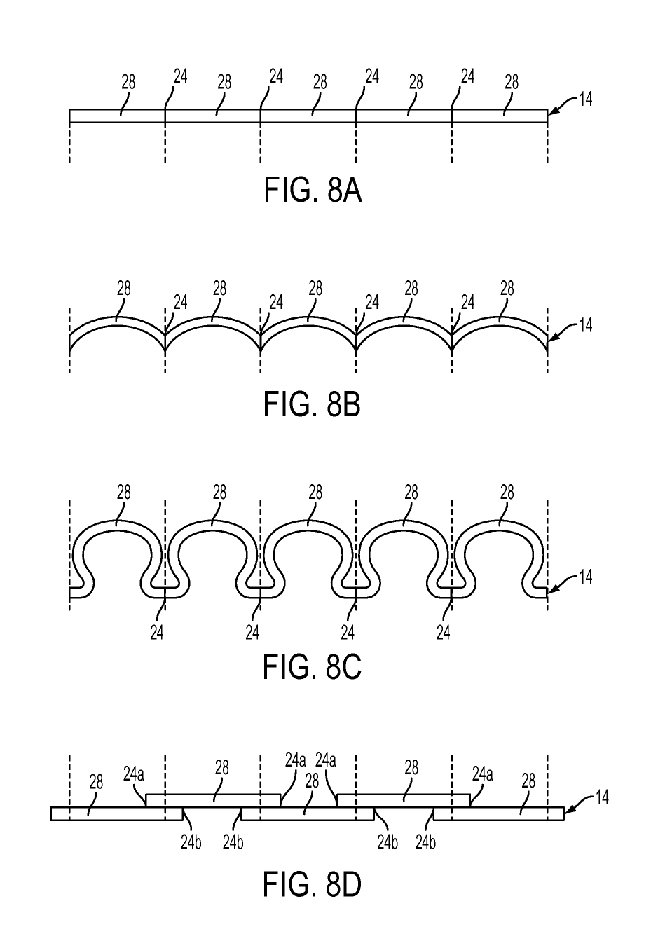

FIG. 8A provides a schematic illustration of an aspect of an embodiment of the neonatal drape device 12 with the objective of showing a side view of a portion of the edge of the sheet 14. Accordingly, as provided in the view are multiple separation lines 24 defining spans 28 there between. Although not shown, it should be appreciated that the illustration could have included other separation lines 26 or spans 30, for example.

FIG. 8B provides a schematic illustration of an aspect of an embodiment of the neonatal drape device 12 of the embodiment shown in FIG. 8A, except for the variation of the spans 28 having a generally curved profile rather than generally flat. The curved span would provide the benefit of additional surface area, for example, when it is re-applied or re-adhered in to the closed or wrapped configuration (after having been separated to create an access passage as per the teachings of the present invention disclosed herein).

FIG. 8C provides a schematic illustration of an aspect of an embodiment of the neonatal drape device 12 of the embodiment shown in FIG. 8A, except for the variation of the spans 28 having a generally horseshoe or mushroom profile rather than generally flat. The horseshoe or mushroom profile would provide the benefit of additional surface area, for example, when it is re-applied or re-adhered in to the closed or wrapped configuration (after having been separated to create an access passage as per the teachings the of invention disclosed herein).

FIG. 8D provides a schematic illustration of an aspect of an embodiment of the neonatal drape device 12 of the embodiment shown in FIG. 8A, except for the variation of the spans 28 having a greater width and is overlapping each other (as defined by separation lines 24a, 24b). The wider span would provide the benefit of additional surface area, for example, when it is re-applied or re-adhered to the closed or wrapped configuration (after having been separated to create an access passage as per the teachings of the present invention disclosed herein).

It should be appreciated that in an embodiment the thickness of the sheet 14 as disclosed herein may be about 1.2 mils, which equates to about 30.48 μm. It should be appreciated that in an embodiment the thickness of the sheet 14 as disclosed herein may be between about 10 μm to about 100 μm. It should be appreciated that in an embodiment the thickness of the sheet 14 as disclosed herein may be between about 20 μm to about 90 μm. It should be appreciated that in an embodiment the thickness of the sheet 14 as disclosed herein may be between about 30 μm to about 80 μm. It should be appreciated that in an embodiment the thickness of the sheet 14 as disclosed herein may be between about 40 μm to about 70 μm. It should be appreciated that in an embodiment the thickness of the sheet 14 as disclosed herein may be between about 50 μm to about 60 μm. It should be appreciated that in an embodiment the thickness of the sheet 14 as disclosed herein may be between about 60 μm to about 100 μm. It should be appreciated that in an embodiment the thickness of the sheet 14 as disclosed herein may be between about 20 μm to about 50 μm. It should be appreciated that in an embodiment the thickness of the sheet may be greater than 100 μm. It should be appreciated that in an embodiment the thickness of the sheet may be less than 10 μm. It should be appreciated that in an embodiment the thickness of the sheet may be about 30.5 μm. It should be appreciated that in an embodiment the thickness of the sheet may be about 15, 16, 17, 18, 19, 20, 21, 22, 23, 24, 25, 26, 27, 28, 29, 30, 31, 32, 33, 34, 35, 36, 37, 38, 39, 41, 42, 43, 44, or 45 μm.

An advantage of an embodiment of the present invention is that the device can be used in conjunction with infants of a variety of sizes or weights. Similarly, the device may be wrapped to a variety of degrees of tightness or closeness; such as loose fitting, tight fitting, or some degree in between as desired or required. As our neonatal drape device has the ability to conform to nearly any geometry or shape, it may be wrapped as tightly or as loosely as required by the user. In an aspect of our embodiment, it should be appreciated that the wrapping of the neonatal drape device may be substantially skin tight or wrapped with significant slack or spacing around the neonatal subject. It should be appreciated that the drawings are schematics and for illustrative purposes only, and these characteristics may not be accurately portrayed for all instances.

An advantage of an embodiment of the present invention device is that the material of the sheet is self adhesive, but it does not necessarily adhere to neonatal infants that are prone to having extremely weak and thin skin. Similarly, an advantage of an embodiment of the present invention is that it does not require extra instruments, fixtures, adhesives, VELCRO, fasteners, structures, or mechanisms to achieve adherence. Moreover, an advantage of an embodiment of the present invention is that it does not require extra fixtures, structures, or mechanisms to provide access to the neonatal subject.

While the illustrations of the various figures depict the separation lines as horizontal, it should be appreciated that the alignment of the separation lines may be horizontal, substantially horizontal, or pitched upward or downward at any angle as desired or required (or any combination thereof). Similarly, while the illustrations tend to show left side and right separation lines being in horizontal alignment, it should be appreciated that any set (left and right) may be offset as well.

It should be appreciated that while the illustrations of the various figures depict the spans as being relatively uniform in width and length, it should be appreciated that the width and length of the spans may vary within the individual spans themselves in a device. Similarly, the width of the different spans may vary within a device. The separation lines may be straight, curved or angled-segments, or any geometry as desired or required.

It should be appreciated that during practice of an embodiment, the user may pull back more than one panel. Moreover, it should be appreciated that while the illustrations of the various figures depict the number of spans to be about five to seven on each side of the sheet, it should also be appreciated that the sheet may contain many more or less spans (less spans being 2-4). The number of spans on each side of the sheet may range form 7-24. It should be appreciated that the number of spans may range from 25-50 on each side as desired or required. It should be appreciated that the number of spans may range from 51-75 on each side as desired or required It should be appreciated that there may be even more spans as desired or required.

It should be appreciated that while the embodiments discussed herein may prefer a single sheet, it is possible to utilize multiple sheet layers or multi-ply or the like.

It should be appreciated that sheet (and its related components) discussed herein may can take on all shapes along the entire continual geometric spectrum of manipulation of x, y and z planes to provide and meet the anatomical and structural demands, as well as operational requirements as contemplated by various embodiments and teachings disclosed herein.

Although not required, it should be appreciated that optionally other instruments, fixtures, adhesives, VELCRO, fasteners, structures, or mechanisms may be added to the various embodiments.

### EXAMPLES

Practice of an aspect of an embodiment (or embodiments) of the invention will be still more fully understood from the following examples and experimental results, which are presented herein for illustration only and should not be construed as limiting the invention in any way.

### Example Set No. 1

An aspect of an embodiment of the present invention provides, among other things, a drape device (and related method of use and manufacture) for extremely low birth weight (ELBW) infants, so that they can be enclosed within the drape device. A purpose of the drape device is to decrease heat/body temperature loss and decrease insensible water loss. The drape device also has perforations throughout so that access can be obtained to the infant subject without compromising all of the heat that has been enclosed.

### Example Set No. 2

An aspect of an embodiment of the present invention neonatal thermoregulation drape device provides a square (or rectangular or other desired or required shapes, such as polygon, circular, oval shape, multi-curvature, etc.) with a hood at or near the center (or other desired or required location) of one of the edges. The drape device is to be placed on the infant warmer table, open. The newborn is to be placed on top of the drape device with his/her head in the hood. The sides of the drape device are then to be wrapped around the newborn. There are perforations along the sides of the drape device so that “spans” can be made in the material. For example, this is so that if access to the umbilicus (or other area or device) is needed, the entire wrap device does not need to be opened, allowing for heat and insensible water loss to occur. There can be a span opened over the chest, abdomen and legs (or other regions) if needed or desired.

### Example Set No. 3

In an approach, an embodiment of the neonatal thermoregulation drape device is provided for the extremely low birth weight (ELBW) infants. A purpose of the device is to decrease heat loss through evaporation and radiation, not only immediately after birth, but it can be utilized to help regulate the infant's temperature until the incubator has reached the desirable temperature and humidity. Its perforations allow for spans to be created so that thermoregulation does not have to be disrupted if umbilical lines (or other devices or instruments) need to be placed for example. If umbilical lines are needed, just the spans over the abdomen can be opened. The hood for the infants head is also convenient to prevent heat loss from the head. The clear material allows for unobstructed and continuous observation for the infant.

### Example Set No. 4

An aspect of various embodiments of the present invention may provide a number of novel and nonobvious features, elements and characteristics, such as but not limited thereto, the following: a) it avoids guessing how a device should be placed on the warmer table, b) it avoids cumbersome placement of devices over the infant's head with a hat while ensuring the hat will not be displaced or cause other inadvertent disruptions, c) it avoids the need for cutting, and d) the perforations allow for openings exactly where you want them so that the infant can be treated/accessed.

An aspect of various embodiments of the present invention may be utilized for a number of products and services, such as but not limited thereto, Neonatal Intensive care units in the country, who care for ELBW patients who would benefit from the present invention. Insensible heat loss through radiation and evaporation are of serious concern, as such the present invention is specifically made to help in decreasing those risks.

### Additional Examples

### Example 1

An aspect of an embodiment of the present invention provides, but not limited thereto, a neonatal drape device for protecting a neonatal subject. The device may comprise: a sheet configured to receive the neonatal subject in a supine position having at least one side edge and a plurality of separation lines; the separation lines extending inwardly from the at least one side edge of the sheet; the sheet being sufficiently pliable so as to wrap and conform to the neonatal subject; the sheet comprised of a material that is repeatably self adhesive as wrapped around the neonatal subject, wherein the repeatable self adhesion is due to characteristics of the material; the separation lines configured to define a span between adjacent the separation lines or between the separation line and the at least one side edge to allow user to selectively separate the span from an adjacent span in its wrapped configuration and form an access passage to the neonatal subject; and the span configured to repeatably self adhere to the sheet in its wrapped configuration to allow the user to selectively close the access passage.

### Example 2

The device of example 1, wherein the material of the sheet comprises a polymer.

### Example 3

The device of example 1 (as well as subject matter of example 2), wherein the material of the sheet is transparent.

### Example 4

The device of example 1 (as well as subject matter of one or more of any combination of examples 2-3), wherein the sheet is sufficiently thin to allow auscultation there through.

### Example 5

The device of example 1 (as well as subject matter of one or more of any combination of examples 2-4), wherein the separation lines are configured to be sufficiently strong to remain un-separated during initial wrapping and to separate upon the user selectively separating the span from the adjacent span in its wrapped configuration.

### Example 6

The device of claim 1 (as well as subject matter of one or more of any combination of examples 2-5), further comprising an appendage disposed on the at least one side edge.

### Example 7

The device of example 7 (as well as subject matter of one or more of any combination of examples 2-6), wherein the appendage provides a hood for the head of the neonatal subject.

### Example 8

The device of example 1 (as well as subject matter of one or more of any combination of examples 2-7), wherein the sheet is configured in a polygonal shape.

### Example 9

The device of example 1 (as well as subject matter of one or more of any combination of examples 2-8), wherein the sheet is configured in a circular or elliptical shape.

### Example 10

The device of example 1 (as well as subject matter of one or more of any combination of examples 2-9), wherein the separation lines comprise at least one of the following: perforations, scores, thin portions, or weak portions.

### Example 11

The device of example 1 (as well as subject matter of one or more of any combination of examples 2-10), wherein the material comprises at least one of the following: polyethylene, polyurethane, vinyl, Poly vinyl chloride (PVC), rubber, or vinyl.

### Example 12

The device of example 1 (as well as subject matter of one or more of any combination of examples 2-11), wherein the material comprises: breathable material, permeable material, impermeable material, nano-porous material, or micro-porous material.

### Example 13

The device of example 1 (as well as subject matter of one or more of any combination of examples 2-12), wherein the access passage accommodates at least one of the following: medical instrument, medical device, material, or fingers or hand of the user.

### Example 14

The device of example 1 (as well as subject matter of one or more of any combination of examples 2-13), wherein the separation lines extend inwardly from two of the side edges.

### Example 15

The device of example 14 (as well as subject matter of one or more of any combination of examples 2-14), wherein the two of the side edges are opposing one another.

### Example 16

The device of example 1 (as well as subject matter of one or more of any combination of examples 2-15), wherein the separation lines extend inwardly from opposing portions of one of the side edge.

### Example 17

The device of example 1 (as well as subject matter of one or more of any combination of examples 2-16), wherein the neonatal subject comprises a neonatal human or neonatal animal.

### Example 18

The device of example 1 (as well as subject matter of one or more of any combination of examples 2-17), wherein the thickness of the sheet is about 30 to about 31 μm.

### Example 19

An aspect of an embodiment of the present invention provides, but not limited thereto, a neonatal drape device for protecting a neonatal subject. The device may comprise: a sheet configured to receive the neonatal subject in a supine position having at least one side edge and a plurality of separation lines; the separation lines extending inwardly from the at least one side edge of the sheet from a first portion and a second portion of the sheet, wherein the first and second portions are generally opposed; the sheet being sufficiently pliable so as to wrap and conform to the neonatal subject; the sheet comprised of a material that is repeatably self adhesive as wrapped around the neonatal subject, wherein the repeatable self adhesion is due to characteristics of the material; wherein for each of the first portion of the sheet and the second portion of the sheet, the separation lines configured to define a span between adjacent the separation lines or between the separation line and the at least one side edge to allow user to selectively separate the span from an adjacent span in its wrapped configuration and form an access passage to the neonatal subject; and wherein for each of the first portion of the sheet and the second portion of the sheet, the span configured to repeatably self adhere to the sheet in its wrapped configuration to allow the user to selectively close the access passage.

### Example 20

The device of example 19 (as well as subject matter of one or more of any combination of examples 2-19), wherein for each of the first portion of the sheet and the second portion of the sheet, the separation lines are configured to be sufficiently strong to remain un-separated during initial wrapping and to separate upon the user selectively separating the span from the adjacent span in its wrapped configuration.

### Example 21

The device of example 19 (as well as subject matter of one or more of any combination of examples 2-19), wherein the thickness of the sheet is about 30 to about 31 μm.

### Example 22

An aspect of an embodiment of the present invention provides, but not limited thereto, a method of protecting a neonatal subject. The method may comprise: providing a sheet to receive the neonatal subject, the sheet having at least one side edge and a plurality of separation lines; placing the neonatal subject in a supine position on the sheet; wrapping the neonatal subject with the sheet so as to conform the sheet to the neonatal subject and adhering portions of the sheet to other portions of the sheet; tearing along the separation lines, after the neonatal subject has been wrapped, releasing a span between the separation lines allowing access to the neonatal subject; and re-applying the span thereby closing the access and re-adhering the span to the sheet.

### Example 23

The method of example 21, further comprising inserting at least one of the following into said access: medical instrument, medical device, material, or fingers or hand of the user.

### Example 24

The method of manufacturing the drape device of any examples 1-21.

The devices, systems, compositions, manufacturing and fabrication techniques, materials, and methods of various embodiments of the invention disclosed herein may utilize aspects disclosed in the following references, applications, publications and patents and which are hereby incorporated by reference herein in their entirety (and which are not admitted to be prior art with respect to the present invention by inclusion in this section):
1. International Patent Application Serial No. WO 2007/141490 A1, Pearson, et al., “Neonatal Swaddler”, Dec. 13, 2007.
2. U.S. Pat. No. 3,739,399, Sheahon, J., “Neonatal Wrap”, Jun. 19, 1973.
3. U.S. Patent Application Publication No. US 2012/0017372 A1, Lamberti, J., “Infant Swaddling Blanket”, Jan. 26, 2012.
4. U.S. Patent Application Publication No. US 2009/0144875 A1, Pearson, et al., “Neonatal Swaddler”, Jun. 11, 2009.
5. U.S. Pat. No. 5,621,917, Howsden, S., “Infant Care Garment”, Apr. 22, 1997.
6. U.S. Pat. No. 3,636,566, Sutherland, J., “Swaddler”, Jan. 25, 1972.
7. U.S. Patent Application Publication No. US 2012/0017372 A1, Lamberti, J., “Infant Swaddling Blanket”, Jan. 26, 2012.
8. U.S. Patent Application Publication No. US 2012/0023637 A1, Neveu, J., “Infant Swaddling System and Method”, Feb. 2, 2012.
9. U.S. Pat. No. 6,640,340 B2, Gibson, L., “Swaddling Cloth”, Nov. 4, 2003.
10. U.S. Pat. No. 5,852,827, Lear, L., “Baby Wrapping Blanket”, Dec. 29, 1998.
11. U.S. Pat. No. 7,254,849 B1, Fiebrich, et al., “Snug & Tug Swaddling Blanket”, Aug. 14, 2007.
12. U.S. Pat. No. 4,616,365, Lyons, S., “Wrap”, Oct. 14, 1986.
13. U.S. Pat. No. 8,191,188 B2, Kaplan, et al., “Swaddle Blanket”, Jun. 5, 2012.
14. U.S. Pat. No. 4,979,250, Troncone, et al., “Blanket for a Newborn Infant”, Dec. 25, 1990.
15. U.S. Pat. No. 5,044,031, Sherwood, et al., “Passive Rewarming Articles”, Sep. 3, 1991.
16. U.S. Pat. No. 5,129,406, Magnusen, et al., “Method for Using an Infant Garment with Crossed Over Arm Positioning Sleeves”, Jul. 14, 1992.
17. U.S. Pat. No. 5,640,717, Ray, C., “Comforting Clothing for Newborns”, Jun. 24, 1997.
18. U.S. Pat. No. 6,928,674 B2, Blackburn, N., “Swaddling Blanket”, Aug. 16, 2005.
19. U.S. Pat. No. 6,393,612 B1, Thach, et al., “Garment”, May 28, 2002.
20. U.S. Pat. No. 7,246,392 B2, Schmid, et al., “Wearable Blanket and a Swaddling Accessory Therefore”, Jul. 24, 2007.
21. U.S. Patent Application Publication No. US 2011/0021886 A1, Briggs, C., Jan. 27, 2011.
22. U.S. Pat. No. 4,083,064, Schneider, K., “Infant Thermal Shield”, Apr. 11, 1978.
23. U.S. Pat. No. 4,611,353, Als, et al., “Infant's Garment”, Sep. 16, 1986.
24. EP No. 2 543 269 A1, Lestoquoy, P., et al., “Cover Neonatal”, Jan. 9, 2013.
25. U.S. Pat. No. 6,210,320 B1, Rogone, et al., “Thermal and Humidity Barrier for Extremely Premature Infants”, Apr. 3, 2001.
26. U.S. Pat. No. 4,897,885, Lunt, A., “One-Piece Infant Bunting”, Feb. 6, 1990.
27. Mance, 2008, “Keeping Infants Warm: Challenges of Hypothermia,” Advances in Neonatal Care, 8(1), 6-12.
28. Miller, Lee, & Gould, 2011, “Hypothermia in Very Low Birth Weight Infants: Distribution, Risk Factors and Outcomes”, Journal of Perinatology, 31, S49-S56).
29. Rohana, Khairina, Boo, & Shareena, 2011, “Reducing Hypothermia in Preterm Infants with Polyethylene Wrap,” Pediatrics International, 53, 468-474, Doi: 10.1111/j.1442-200X.2010.03295.x)

Moreover, for example, any of the methods of manufacturing the devices or items (or portions thereof) or as disclosed in the references, applications, publications and patents as disclosed in the above references (nos. 1-29), as well as techniques of manufacturing available to one skilled in the art are also incorporated by reference and may therefore be considered as part of the present invention and employed within the context of the invention (and which are not admitted to be prior art with respect to the present invention by inclusion in this section).

Unless clearly specified to the contrary, there is no requirement for any particular described or illustrated activity or element, any particular sequence or such activities, any particular size, speed, material, duration, contour, dimension or frequency, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Moreover, any activity or steps can be done in various orders, as well as sequentially, simultaneously or some combination thereof. It should be appreciated that aspects of the present invention may have a variety of sizes, contours, shapes, compositions and materials as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions may have been provided on the aforementioned figures, the device may constitute various sizes, dimensions, contours, rigidity, shapes, flexibility and materials as it pertains to the components or portions of components of the device, and therefore may be varied and utilized as desired or required.

It should be appreciated that the related components or portions of the related components as discussed herein may take on all shapes along the entire continual geometric spectrum of manipulation of x, y and z planes to provide and meet the anatomical and structural demands, operational requirements, and surgical needs.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

I claim:

1. A neonatal drape device for protecting a neonatal subject, said device comprising:
a sheet configured to receive the neonatal subject in a supine position having at least one side edge and a plurality of separation lines;
said separation lines extending inwardly from said at least one side edge of said sheet;
said sheet being sufficiently pliable so as to wrap and conform to the neonatal subject;
said sheet comprised of a material that is repeatably self adhesive as wrapped around the neonatal subject, wherein said repeatable self adhesion is due to characteristics of said material;
said separation lines configured to define a span between adjacent said separation lines or between said separation line and said at least one side edge to allow user to selectively separate said span from an adjacent span in its wrapped configuration and form an access passage to the neonatal subject; and
said span configured to repeatably self adhere to said sheet in its wrapped configuration to allow the user to selectively close said access passage.

2. The device of claim 1, wherein said material of said sheet comprises a polymer.

3. The device of claim 1, wherein said material of said sheet is transparent.

4. The device of claim 1, wherein said sheet is sufficiently thin to allow auscultation there through.

5. The device of claim 1, wherein said separation lines are configured to be sufficiently strong to remain un-separated during initial wrapping and to separate upon the user selectively separating said span from said adjacent span in its wrapped configuration.

6. The device of claim 1, further comprising an appendage disposed on said at least one side edge.

7. The device of claim 7, wherein said appendage provides a hood for the head of the neonatal subject.

8. The device of claim 1, wherein said sheet is configured in a polygonal shape.

9. The device of claim 1, wherein said sheet is configured in a circular or elliptical shape.

10. The device of claim 1, wherein said separation lines comprises at least one of the following: perforations, scores, thin portions, or weak portions.

11. The device of claim 1, wherein said material comprises at least one of the following: polyethylene, polyurethane, vinyl, Poly vinyl chloride (PVC), rubber, or vinyl.

12. The device of claim 1, wherein said material comprises: breathable material, permeable material, impermeable material, nano-porous material, or micro-porous material.

13. The device of claim 1, wherein said access passage accommodates at least one of the following: medical instrument, medical device, material, or fingers or hand of the user.

14. The device of claim 1, wherein said separation lines extend inwardly from two of said side edges.

15. The device of claim 14, wherein said two of said side edges are opposing one another.

16. The device of claim 1, wherein said separation lines extend inwardly from opposing portions of one of said side edge.

17. The device of claim 1, wherein said neonatal subject comprises a neonatal human or neonatal animal.

18. The device of claim 1, wherein the thickness of the sheet is about 30 to about 31 μm.

19. A neonatal drape device for protecting a neonatal subject, said device comprising:
a sheet configured to receive the neonatal subject in a supine position having at least one side edge and a plurality of separation lines;
said separation lines extending inwardly from said at least one side edge of said sheet from a first portion and a second portion of said sheet, wherein said first and second portions are generally opposed;
said sheet being sufficiently pliable so as to wrap and conform to the neonatal subject;
said sheet comprised of a material that is repeatably self adhesive as wrapped around the neonatal subject, wherein said repeatable self adhesion is due to characteristics of said material;
wherein for each of said first portion of said sheet and said second portion of said sheet, said separation lines configured to define a span between adjacent said separation lines or between said separation line and said at least one side edge to allow user to selectively separate said span from an adjacent span in its wrapped configuration and form an access passage to the neonatal subject; and
wherein for each of said first portion of said sheet and said second portion of said sheet, said span configured to repeatably self adhere to said sheet in its wrapped configuration to allow the user to selectively close said access passage.

20. The device of claim 19, wherein for each of said first portion of said sheet and said second portion of said sheet, said separation lines are configured to be sufficiently strong to remain un-separated during initial wrapping and to separate upon the user selectively separating said span from said adjacent span in its wrapped configuration.

21. The device of claim 19, wherein the thickness of the sheet is about 30 to about 31 μm.

22. A method of protecting a neonatal subject, said method comprising:
providing a sheet to receive the neonatal subject, said sheet having at least one side edge and a plurality of separation lines;
placing the neonatal subject in a supine position on said sheet;
wrapping said neonatal subject with said sheet so as to conform said sheet to said neonatal subject and adhering portions of said sheet to other portions of said sheet;
tearing along said separation lines, after said neonatal subject has been wrapped, releasing a span between said separation lines allowing access to said neonatal subject; and
re-applying said span thereby closing said access and re-adhering said span to said sheet.

23. The method of claim 21, further comprising inserting at least one of the following into said access: medical instrument, medical device, material, or fingers or hand of the user.
